(19) United States
(12) Patent Application Publication
(10) Pub. No.: US 2010/0112697 A1
(43) Pub. Date: May 6, 2010

(54) PROCESS FOR THE HIGH-PURITY ISOLATION OF MESENCHYMAL STEM CELLS DERIVED FROM PLACENTAL CHORIONIC PLATE MEMBRANE

(75) Inventors: Gi-Jin Kim, Seoul (KR); Kyung-Sun Shin, Gyeonggi-do (KR); Kyu-Hwan Na, Incheon (KR); Hyung-Min Chung, Gyeonggi-do (KR)

Correspondence Address: THE NATH LAW GROUP, 112 South West Street, Alexandria, VA, 22314, US

(73) Assignees: CHABIO & DIOSTECH CO., LTD., Yongin-si, Gyeonggi-do (KR); COLLEGE OF MEDICINE POCHON CHA UNIVERSITY INDUSTRY, Pochon-siu, Gyeonggi-do (KR)

(21) Appl. No.: 12/451,705

(22) PCT Filed: Oct. 24, 2007
(86) PCT No.: PCT/KR2007/005235

§ 371 Date: Nov. 25, 2009

(30) Foreign Application Priority Data
May 29, 2007 (KR) 10-2007-0052215

(51) Int. Cl. C12N 5/0775

(52) U.S. Cl. 435/381

(57) ABSTRACT

The present invention provides a method for isolating mesenchymal stem cells derived from a placental chorionic plate membrane, the method including: (a) harvesting a chorionic plate membrane from a detached placenta; (b) harvesting cells present in the chorionic plate membrane obtained in step (a) by scraping; (c) adding a solution containing trypsin and ethylenediaminetetraacetate to the cells obtained in step (b) to perform an enzymatic reaction and adding a fetal bovine serum thereto to terminate the enzymatic reaction; and (d) centrifuging the reaction solution obtained in step (c) and culturing the obtained cells in a medium containing a fetal bovine serum and an antibiotic.

### TECHNICAL FIELD

The present invention relates to a method for isolating mesenchymal stem cells derived from a placental chorionic plate membrane at high purity.

### BACKGROUND ART

Mesenchymal stem cells are multipotent stem cells that have self-renewal capacity and can differentiate into various lineages. Mesenchymal stem cells are also called “mesenchymal progenitor cells”. Mesenchymal stem cells can differentiate into is bone, fat, cartilage, nerve, muscle, bone marrow stromal cells, etc. according to conditions, and thus, have various therapeutic efficacies. Mesenchymal stem cells are a kind of adult stem cells, and can be isolated together with hematopoietic stem cells from the bone marrow. Mesenchymal stem cells are adhered to culture dishes, unlike hematopoietic stem cells that are floating in culture dishes. Mesenchymal stem cells have been used in various experiments and/or clinical applications. However, the differentiation capacity of bone marrow-derived mesenchymal stem cells is reduced with the donor's age, and the number of mesenchymal stem cells to be isolated significantly varies according to the donor's conditions. And also, during the extraction and isolation of bone marrow-derived mesenchymal stem cells, donors may suffer from pains. Thus, there is an increasing need to develop a new method of isolating mesenchymal stem cells.

Meanwhile, a placenta is now routinely discarded after birth. However, it has been found that various cells such as mesenchymal stem cells, decidua cells, trophoblast cells, amniotic cells, and endothelial cells are present in portions of the placenta. The Tsuji group and the Kanhai group have reported that there is a higher likelihood that placenta-derived cells are mesenchymal stem cells and progenitor cells (Stem Cells 2004; 22(5): 649-58; Stem Cells 2004; 22(7):1338-1345), and the Surbek group has suggested that there is a likelihood of self-immune transplantation of placenta-derived mesenchymal stem cells for neuronal regeneration (Am J Obstet Gynecol 2006; 194(3):664-673). The Takahashi group has reported the cartilage regeneration potential of human placenta-derived mesenchymal stem cells (Biochem Biophys Res Commun 2006; 340(3):944-952).

Considering common limitations to the research and utilization of stem cells, i.e., ethical issues, the limited number of cells to be isolated, and the types of stem cells that can be isolated from limited single tissues, it is very important to establish a method of isolating placenta-derived stem cells. Although the potentials of various stem cells derived from the placenta were identified as described above, there is a problem in that it is difficult to isolate mesenchymal stem cells, at high purity, from a mixture of various other cells present in the placenta.

Zhang et al. disclose a method of isolating mesenchymal progenitor cells from placenta and the characteristics of the isolated mesenchymal progenitor cells (Experimental Hematology 32 (2004) 657-664). According to this method, amniotic sac and decidua are removed from placenta. Then, the placenta is washed with a phosphate buffered saline, and an irrigating solution and an culture solution (lscove's modified Dulbecco medium supplemented with heparin 12.5 U/ml, penicillin 50 U/ml, and streptomycin 50 mg/ml) are allowed to flow through arterial-vein circuit to remove residual blood from the tissues. The tissues are immersed in the culture solution for 12 to 24 hours, and mononuclear cells are obtained using a Ficoll density gradient and resuspended in a fetal bovine serum (FBS)-containing medium to thereby obtain mesenchymal progenitor cells. The method can be performed at a laboratory scale, but it requires complicated procedures, including Ficoll density-gradient separation. Moreover, since mesenchymal stem cells are cultured in the placenta itself for a long time, mixing of mononuclear cells present in the placenta may be caused. In addition, it is difficult to stably isolate/purify a large amount of healthy mesenchymal stem cells, making it difficult to clinically apply the mesenchymal stem cells. Still furthermore, since placenta free from amniotic sac and decidua is used, the purity of mesenchymal progenitor cells may be lowered since the mesenchymal progenitor cells can be mixed with other cells derived from placental villi.

Recently, S. J. Kim et al. disclose a method of promoting hematopoietic differentiation of embryonic stem cells by isolating mesenchymal stem cells from a placental chorionic plate membrane and co-culturing the mesenchymal stem cells with embryoid bodies formed from the embryonic stem cells (Acta Haematol 2006; 116; 219-222). According to the method, the isolation of the mesenchymal stem cells from the placental chorionic plate membrane is performed by isolating the chorionic plate membrane from the placenta, isolating cells from the chorionic plate membrane by an enzyme treatment, and culturing the cells in a DMEM supplemented with 20% FBS and antibiotic(s). The method can relatively easily isolate/purify mesenchymal stem cells, but amniotic cells attached to the chorionic plate membrane, in addition to the mesenchymal stem cells, are isolated due to the enzyme treatment of the entire surface of the chorionic plate membrane, thereby causing lowered cell purity. Therefore, when mesenchymal stem cells are sub-cultured for several passages, they are mixed with cells derived from the amnion. The amniotic cells are proliferated rapidly than the mesenchymal stem cells, making the pure culture of the mesenchymal stem cells difficult. In addition, the mesenchymal stem cells are susceptible to an enzymatic reaction. When an enzyme treatment is performed at 37° C., the membranes of the mesenchymal stem cells are easily damaged, thereby adversely affecting cell viability. Therefore, this conventional method of isolating mesenchymal stem cells derived from a placental chorionic plate membrane involves a problem of mixing with other cells.

### DETAILED DESCRIPTION OF THE INVENTION

### Technical Problem

While endeavoring to develop a method for isolating mesenchymal stem cells derived from the placenta at high purity while avoiding ethical problems associated with stem cells, the present inventors have found that when cells are harvested from a placental chorionic plate membrane using a physical scraping method and subjected to enzyme treatment under mild conditions and separate enzymatic reaction termination, mesenchymal stem cells can be isolated at high purity and viability.

Therefore, the present invention provides a method for isolating mesenchymal stem cells derived from a placental chorionic plate membrane at high purity.

### Technical Solution

According to an aspect of the present invention, there is provided a method for isolating mesenchymal stem cells derived from a placental chorionic plate membrane, the method including: (a) harvesting a chorionic plate membrane from a detached placenta; (b) harvesting cells present in the chorionic plate membrane obtained in step (a) by scraping; (c) adding a solution containing trypsin and ethylenediaminetetraacetate (EDTA) to the cells obtained in step (b) to perform an enzymatic reaction and adding a fetal bovine serum thereto to terminate the enzymatic reaction; and (d) centrifuging the reaction solution obtained in step (c) and culturing the obtained cells in a medium containing a fetal bovine serum and an antibiotic.

### ADVANTAGEOUS EFFECTS

According to the present inventive isolation method, cells are harvested from the inner portion of a placental chorionic plate membrane by a physical scraping method. Therefore, other cells (including amniotic cells) which are attached to the inner portion of the chorionic plate membrane can be excluded, thereby increasing the purity of mesenchymal stem cells. Moreover, an enzymatic reaction is performed under mild conditions, and an enzymatic reaction termination is separately performed, thereby significantly increasing cell viability. Therefore, the present inventive isolation method enables high-purity isolation and large-scale proliferation of mesenchymal stem cells, and thus, can be used for various stem cell therapies, e.g., cell therapy in the treatment of degenerative diseases, etc.

### BRIEF DESCRIPTION OF THE DRAWINGS

FIGS. 1 through 3 respectively show the morphology, karyotype, and cell cycle of placental chorionic plate membrane-derived mesenchymal stem cells obtained according to the present inventive isolation method;

FIG. 4 shows fluorescence activated cell sorting (FACS) analysis results of placental chorionic plate membrane-derived mesenchymal stem cells obtained according to the present inventive isolation method; and

FIG. 5 shows RT-PCR analysis results of genes expressed from placental to chorionic plate membrane-derived mesenchymal stem cells obtained according to the present inventive isolation method.

### BEST MODE FOR CARRYING OUT THE INVENTION

According to the present inventive isolation method, cells are harvested from the inner portion of a placental chorionic plate membrane by a physical scraping method. Therefore, other cells (including amniotic cells) which are attached to the inner portion of the chorionic plate membrane can be excluded, thereby increasing the purity of mesenchymal stem cells. Moreover, an enzymatic reaction is performed under mild conditions, and an enzymatic reaction termination is separately performed, thereby significantly increasing cell viability. Therefore, the present inventive isolation method enables high-purity isolation and large-scale proliferation of mesenchymal stem cells, and thus, can be used for various cell therapies, e.g., cell therapy in the treatment of degenerative diseases, etc.

A present inventive isolation method includes harvesting a chorionic plate membrane from a detached placenta [step (a)]. The detached placenta may be a placenta separated and discarded from a healthy woman after birth. That is, the “detached placenta” refers to a placenta separated from the body of a woman after birth. The detached placenta may be promptly stored in a sterilized bag placed in an ice bath. The harvesting of the chorionic plate membrane from the detached placenta may be performed by a conventional anatomical method, e.g., by pulling and peeling the chorionic plate membrane surrounding the fetal side of the placenta. The chorionic plate membrane thus obtained are washed twice or more, preferably five times, with an antibiotic (e.g., penicillin, stereptomycin)-containing phosphate buffered saline (PBS), to remove contaminants present in the tissues.

The present inventive isolation method includes harvesting cells by scraping the inner portion of the chorionic plate membrane [step (b)]. The scraping may be performed by means of a tool capable of scraping cells, e.g., a sterilized slide glass or a scraper for cell culture. Preferably, the scraping may be performed by scraping the maternal side of the chorionic plate membrane using a sterilized slide glass. In detail, the chorionic plate membrane is spread on a glass dish so that amniotic cells face down, and cells present in the inner side of the chorionic plate membrane are physically separated and harvested by means of a sterilized slide glass.

The harvested cells may be directly treated with an enzyme. Alternatively, the cells may be washed with an appropriate buffer, concentrated by centrifugation, and then treated with an enzyme. The washing may be performed twice or three times using a buffer such as HBSS (Hank's balanced salt solution), and the centrifugation may be performed at 1000 to 1200 rpm for about 5 to 10 minutes, preferably at about 1,000 rpm for about 5 minutes.

The enzyme treatment of step (c) may be performed using a solution containing trypsin and ethylenediaminetetraacetate (EDTA). The concentrations of trypsin and EDTA are not particularly limited. For example, a 0.25% trypsin/EDTA solution may be used. Step (c) further includes an enzymatic reaction termination process. That is, in the present inventive isolation method, the enzymatic reaction is terminated by addition of fetal bovine serum (FBS) to minimize cell damage by enzymes.

In the present inventive isolation method, the enzyme treatment and the enzymatic reaction termination may be twice repeated to increase the yield of mesenchymal stem cells. That is, the enzyme treatment and the enzymatic reaction termination may be performed once or twice. When the enzyme treatment and the enzymatic reaction termination are performed once, the enzyme treatment may be continued for about one hour. When the enzyme treatment and the enzymatic reaction termination are performed twice, each enzyme treatment may be continued for about 30 minutes.

In the present inventive isolation method, the enzyme treatment may be gradually performed at a relatively low temperature, e.g., at about 20 to 30° C., preferably at room temperature, unlike a conventional enzyme treatment at about 37° C. By doing so, cell damage can be significantly reduced.

The present inventive isolation method includes centrifuging the solution obtained in step (c) and culturing the harvested cells in a mesenchymal stem cell culture medium, e.g., in a medium containing a fetal bovine serum (FBS) and antibiotic(s) [step (d)]. The centrifugation may be performed at about 1,000 rpm for about 5 minutes. The mesenchymal stem cell culture medium may be a medium containing a relatively small amount (e.g., 5 to 10%, preferably about 10%) of FBS, e.g., DMEM/F12 supplemented with 10% FBS, 1% penicillin-streptomycin, 1 ug/ml heparin, and 25 ng/ml fibroblast growth factor-4 (FGF-4). The culturing may be performed under conventional culture conditions, e.g., at 37° C. in a CO₂ incubator.

Hereinafter, the present invention will be described more specifically with reference to the following examples. The following examples are for illustrative purposes only and are not intended to limit the scope of the invention.

### Example 1

### Isolation of Mesenchymal Stem Cells

After an informed consent form was signed by a healthy woman who had no medical, obstetrical and surgical problems and delivered a normal child (with no deformation and multiple fetuses) at 37 weeks' gestation or more, a normal placenta was obtained from the woman, and promptly stored in a sterilized bag placed in an ice bath. Then, the morphological and structural characteristics of the placenta was visually observed and recorded. The chorionic plate membrane surrounding the fetus side of the placenta was pulled, peeled, and washed five times with an antibiotic (1% penicillin and streptomycin)-containing PBS buffer to remove contaminants that might be generated in the tissues during cell collection and transportation.

The chorionic plate membrane was spread on a sterilized glass plate with a diameter of 150 mm so that the amniotic cells faced down, and cells of the mesenchymal stem cell layer present in the inner portion of the chorionic plate membrane were scraped and collected with a sterilized slide glass. The collected cells were washed three times with a sterilized HBSS solution and centrifuged at 1,000 rpm for 5 minutes. After removing the supernatant, 10 ml of a 0.25% trypsin/EDTA solution was added to the residual cells. The mixture was incubated at room temperature for 30 minutes while gradually stirring, and 1 ml of FBS was added thereto to terminate the enzymatic reaction. The supernatant (a primary enzymatic reaction solution) was removed and transferred to a 50 ml conical tube. 10 ml of a 0.25% trypsin/EDTA solution was added to the residual cells, and the mixture was incubated at room temperature for 30 minutes while gradually stirring. The supernatant was removed and combined with the primary enzymatic reaction solution. 2.5 ml of FBS was added to the resultant enzymatic reaction solution to terminate the enzymatic reaction, and the resultant solution was centrifuged at 1,000 rpm for 5 minutes. The supernatant was removed and cells were harvested. The cells were added to 3 ml of a culture medium (DMEM/F12 supplemented with 10% FBS, 1% penicillin-streptomycin, 1 ug/ml heparin, and 25 ng/ml FGF-4). The culture solution was sufficiently stirred, transferred to a T25 flask, and incubated at 37° C. in a CO₂ incubator.

Cell isolation was initiated on Sep. 4, 2006, and the cells were subcultured for 10 passages (once per about five days) considering the growth rate of the cells. Cells obtained after the 10 passages were designated “CHA-PDMSC-1”.

### Example 2

### Analysis of Morphological Characteristics

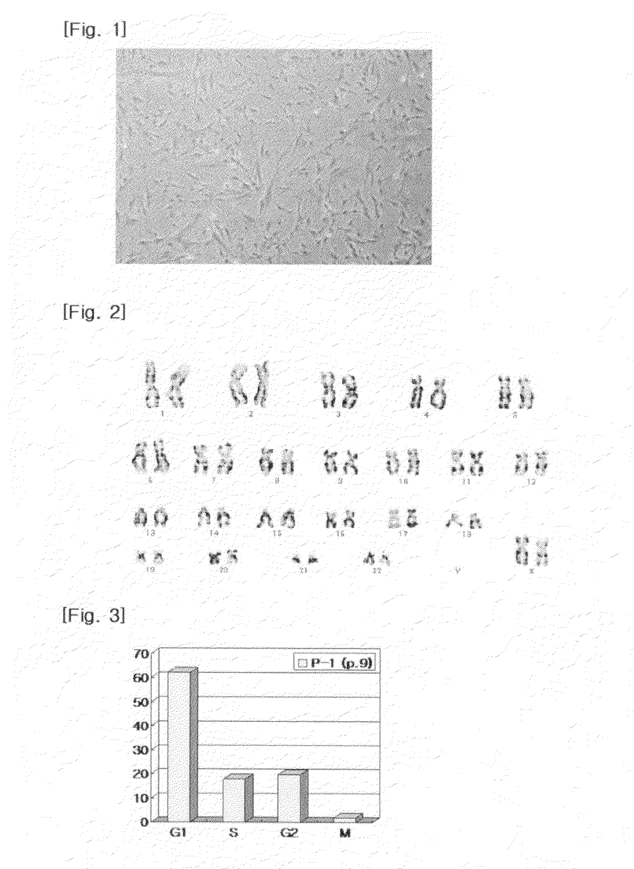

The cells (CHA-PDMSC-1) obtained in Example 1 were observed with a phase contrast microscope, and the result is shown in FIG. 1. The morphology of CHA-PDMSC-1 shows fibroblastoid type. In a Mycoplasma test using a Mycoplasma detection kit (iNtRON Biotechnology, Inc.), the cells were found to be negative. The cells were treated with colcemid (Invitrogen) and KCl solution (0.075M KCl), and stained with Trypsin-Giemsa, and the karyotypes of the cells were determined using CytoVision (Applied Imaging). As a result, the karyotypes of the cells were 46 XX (see FIG. 2). In addition, the cells were stained with propidium iodide (PI), and the cell cycle of the cells was measured using a flow cytometer (FACS, Beckman). As a result, the cells showed a faster than normal cell cycle (see FIG. 3).

The above results show that the cells obtained in Example 1 are new, safe and healthy cells that are Mycoplasma negative, have normal karyotypes, and show a rapid cell division.

### Example 3

### Fluorescence Activated Cell Sorting (FACS) Analysis

In order to identify specific antigens present on surfaces of the cells obtained in Example 1 using various antibodies, fluorescence activated cell sorting (FACS) analysis was performed. That is, when the cells were grown to 80% confluence, 1 ml of a cell dissociation buffer (GIBCO) was added to the cells to dissociate the cells from the culture tube. The cells were incubated with green or blue fluorescent material-labeled human specific antibodies, i.e., anti-CD13, anti-CD71, anti-CD178, anti-CD44, anti-CD105, anti-CD90, anti-CD95, CD34, anti-CD31, anti-CD33, anti-CD56, anti-CD51, anti-HLA-ABC, anti-HLA-DR, and anti-cytokeratin 7 at room temperature for one hour and washed three times with PBS. FACS analysis was performed using a flow cytometer, and the results are shown in FIG. 4.

As shown in FIG. 4, the cells isolated in Example 1 according to the present inventive isolation method were determined to be CD13 positive (99.98), CD71 positive (≧68.92), CD178 negative (≦4.58), CD44 positive (≧99.98), CD105 positive (≧35.75), CD90 positive (≧99.46), CD95 positive (≧99.98), CD34 negative (≦1.48), CD31 negative (≦1.34), CD33 negative (≦1.37), CD56 positive (≧20.04), CD51 negative (≦58.75), HLA-ABC positive (≧99.55), HLA-DR negative (≦4.19), and cytokeratin 7 negative (≦2.80). That is, the cells obtained in Example 1 expressed antigens specific for mesenchymal stem cells other than vascular endothelial cells, blood cells, and amniotic cells. This result shows that the cells isolated in Example 1 according to the present inventive isolation method are cells having the characteristics of mesencymal stem cells.

### Example 4

### Analysis of RNA Expression Levels of Stem Cell-Associated Genes

RT-PCR was performed for genes expressed from the cells obtained in Example 1 according to the present inventive isolation method. That is, when the cells obtained in Example 1 were grown to about 80% confluence in a T25 flask, the cells were collected and RT-PCR was performed as follows. That is, the cells were lysed with Trizol to extract total RNA. cDNAs were synthesized from the total RNA using reverse transcriptase, and PCR was performed using cDNA-specific primers and Tag DNA polymerase. The PCR products were subjected to electrophoresis on agarose gel to identify the amplified genes. The primer sequences, the composition of a PCR solution, to and the conditions of PCR are summarized in Tables 1 to 3 below.

TABLE 1

| | SEQ. | | Tm | |
|---|---|---|---|---|
| | ID. | | (° | Size |
| Gene | No. | Sequence | C.) | (bp) |
| nanog | 1 | F: TTC TTG ACT GGG ACC TTG TC | 54 | 200 |
| | 2 | R: GCT TGC CTT GCT TTG AAG CA | | |
| sox2 | 3 | F: GGG CAG CGT GTA CTT ATC CT | 52 | 200 |
| | 4 | R: AGA ACC CCA AGA TGC ACA AC | | |
| h AFP | 5 | F: GCT TCG CTT TGC CAA TGC TT | 55 | 500 |
| | 6 | R: ATG CTG CAA ACT GAC CAC GC | | |
| h NF- | 7 | F: TTT CCT CTC CTT CTT CTT CAC | 58 | 700 |
| 68kd | | CTT C | | |
| | 8 | R: GAG TGA AAT GGC ACG ATA CCT | | |
| | | A | | |
| beta | 9 | F: TCC TTC TGC ATC CTG TCA GCA | 58 | 300 |
| actin | 10 | R: CAG GAG ATG GCC ACT GCC GCA | | |

TABLE 2

| PCR Solution | Volume | |
|---|---|---|
| cDNA | 2~3 | ul |
| 10 X h-taq bfr | 2.5 | ul |
| 10 mM dNTP mix | 0.5 | ul |
| Primer 1 (10 pmol) | 1 | ul |
| Primer 2 (10 pmol) | 1 | ul |
| 5 X Band doctor | 0 (X0) or 2.5(X0.5) | ul |
| h-Taq.(2.5U/ul) | 0.25 | ul |
| D.W-DEPC | X | ul |
| Total | 25 | ul |

TABLE 3

| | 95° C. | | Tm ° C. | 72° C. | Cycle |
|---|---|---|---|---|---|
| Denature | 15 | min | | | 1 |
| Amplification | 20 | sec | 40 sec | 1 min | 40 |
| Final | | | | 5 min | 1 |

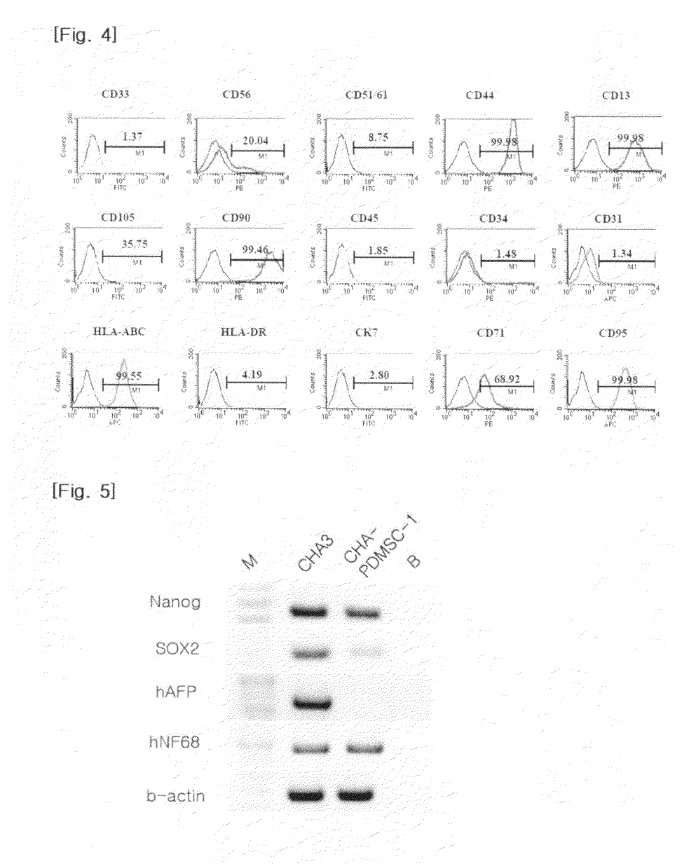

The results of RT-PCR are shown in FIG. 5. As shown in FIG. 5, Nanog and Sox2, which were known to be genes associated with self-renewal of stem cells, were expressed in the cells isolated in Example 1. The expression of NF68 gene, which was a neuroectodermal marker, was also observed. These results show that the cells isolated in Example 1 according to the present inventive isolation method have the characteristics of multipotent stem cells which can be self-renewal and express genes associated with differentiation of neuronal cells in the ectoderm.

### Example 5

### Determination of Teratoma-Forming Potential

The cells (CHA-PDMSC-1) obtained in Example 1 was inserted into the testis capsules of SCID mice (1×10⁶ cells/mouse), and incubated for 12 weeks to determine the incidence of teratoma formation. As a result, no teratoma formation was observed. This result shows that unlike embryonic stem cells forming benign tumors, i.e., teratomas, mesenchymal stem cells obtained the present inventive isolation method are adult stem cells owing to no formation of teratoma, and thus, can be used for safe various cell therapy.

### Example 6

### Determination of Function of Mesenchymal Stem Cells Isolated According to the Present Inventive Isolation Method as Human Feeder Cells for Culturing Human Embryonic Stem Cells

Human embryonic stem cells are pluripotent stem cells that can differentiate into various cells, and thus, have been expected to be used as a cell therapeutic agent for the treatment of degenerative diseases. Most of human embryonic stem cells that have now been established are significantly affected by feeder cells. Currently, feeder cells such as mouse-derived STO cells or MEF (mouse embryonic fibroblasts) have been used. However, since the risk of cross-species contamination must be considered in the development of stem cell therapy, there is an increasing need to develop human-derived feeder cells. In this regard, the use of the cells (CHA-PDMSC-1) isolated in Example 1 according to the present inventive isolation method as human feeder cells was evaluated.

In order to test the feeder cell potential of the CHA-PDMSC-1 cells using the CHA-9 human embryonic stem cells established in the CHA Hospital, one day before subculture of the CHA-9 human embryonic stem cells, the CHA-PDMSC-1 cells were treated with 10 ug/ml of mitomycin C (Sigma) for two hours to set the cell cycle to a non-dividing state called “G0”, and then treated with trypsin/EDTA to isolate the cells. The isolated cells were seeded in a 4-well culture dish (0.8×10⁵ cells/well) and cultured. The next day, the CHA-9 human embryonic stem cells were isolated and placed on the CHA-PDMSC-1 cells that had been attached and cultured in the 4-well culture dish, and the CHA-9 human embryonic stem cells and the CHA-PDMSC-1 cells were co-cultured. During the co-culture, the culture state of the CHA-9 embryonic stem cells was observed in terms of cell morphology, the expression of embryonic stem cell-specific markers, etc. As a result, even when the cells were co-cultured for 10 passages or more, the undifferentiated state and growth rate of the CHA-9 human embryonic stem cells and the expression of the embryonic stem cell-specific markers were favorably maintained.

1. A method for isolating mesenchymal stem cells derived from a placental chorionic plate membrane, the method comprising:
(a) harvesting a chorionic plate membrane from a detached placenta;
(b) harvesting cells present in the chorionic plate membrane obtained in step (a) by scraping;
(c) adding a solution containing trypsin and ethylenediaminetetraacetate to the cells obtained in step (b) to perform an enzymatic reaction and adding a fetal bovine serum thereto to terminate the enzymatic reaction; and
(d) centrifuging the reaction solution obtained in step (c) and culturing the obtained cells in a medium containing a fetal bovine serum and an antibiotic.

2. The method of claim 1, wherein step (b) is performed by scraping a maternal side of the chorionic plate membrane by means of a sterilized slide glass.

3. The method of claim 1, wherein between steps (b) and (c), the cells are washed and concentrated by centrifugation at 1000 rpm for 5 minutes.

4. The method of claim 1, wherein step (c) is twice repeated.

5. The method of claim 4, wherein in step (c), each enzymatic reaction is performed for 30 minutes.

6. The method of claim 1, wherein in step (c), the enzymatic reaction is performed at 20 to 30° C.

7. The method of claim 6, wherein in step (c), the enzymatic reaction is performed at room temperature.

8. The method of claim 1, wherein in step (d), the medium is DMEM/F12 supplemented with 10% fetal bovine serum, 1% penicillin-streptomycin, 1 ug/ml heparin, and 25 ng/ml fibroblast growth factor-4 (FGF-4).

9. The method of claim 2, wherein in step (c), the enzymatic reaction is performed at 20 to 30° C.

10. The method of claim 3, wherein in step (c), the enzymatic reaction is performed at 20 to 30° C.

11. The method of claim 4, wherein in step (c), the enzymatic reaction is performed at 20 to 30° C.

12. The method of claim 5, wherein in step (c), the enzymatic reaction is performed at 20 to 30° C.


SEQUENCE LISTING (10 sequences)

SEQ ID NO: 1 - DNA, 20 bp, Artificial Sequence
  Forward primer
ttcttgactgggaccttgtc

SEQ ID NO: 2 - DNA, 20 bp, Artificial Sequence
  Reverse primer
gcttgccttgctttgaagca

SEQ ID NO: 3 - DNA, 20 bp, Artificial Sequence
  Forward primer
gggcagcgtgtacttatcct

SEQ ID NO: 4 - DNA, 20 bp, Artificial Sequence
  Reverse primer
agaaccccaagatgcacaac

SEQ ID NO: 5 - DNA, 20 bp, Artificial Sequence
  Forward primer
gcttcgctttgccaatgctt

SEQ ID NO: 6 - DNA, 20 bp, Artificial Sequence
  Reverse primer
atgctgcaaactgaccacgc

SEQ ID NO: 7 - DNA, 25 bp, Artificial Sequence
  Forward primer
tttcctctccttcttcttcaccttc

SEQ ID NO: 8 - DNA, 22 bp, Artificial Sequence
  Reverse primer
gagtgaaatggcacgataccta

SEQ ID NO: 9 - DNA, 21 bp, Artificial Sequence
  Forward primer
tccttctgcatcctgtcagca

SEQ ID NO: 10 - DNA, 21 bp, Artificial Sequence
  Reverse primer
caggagatggccactgccgca